(19) United States
(12) Patent Application Publication
(10) Pub. No.: US 2007/0126319 A1
(43) Pub. Date: Jun. 7, 2007

(54) Laboratory cart with removable/slidable storage units

(76) Inventors: Thomas E. Bauer, Saginaw, MI (US); Robert E. Hacker, Farwell, MI (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 11/292,837

(22) Filed: Dec. 2, 2005

(51) Int. Cl. A61B 19/02

(52) U.S. Cl. 312/209

(57) ABSTRACT

A laboratory furniture unit that provides improved flexibility and adjustability in the positioning and reconfiguration of laboratory stations includes a frame defining horizontally spaced apart rails for supporting at least one storage unit, a laboratory bench supported on the frame, and at least one storage unit slidably and/or releasably supported on the rails and below the bench. The laboratory furniture unit facilitates rapid modification of laboratory work space as projects, personnel and equipment changes occur, thus allowing reconfiguration of laboratory work space in minutes, and providing improved productivity, adjustability and optimal ergonomics.

### FIELD OF THE INVENTION

This invention relates to laboratory furniture, and more particularly to portable laboratory furniture having reconfigurable storage units.

### BACKGROUND OF THE INVENTION

Laboratory furniture has typically comprised cabinetry and other storage units, and laboratory bench tops or work surfaces that are permanently installed as fixtures in a laboratory. In general, rearrangement of the furniture and/or reconfiguration of laboratory furniture has required a very substantial effort. Reconfiguration has often involved considerable time, effort and labor to dismantle the furniture and separate the furniture from a building structure, repair damage to the furniture and/or building structure caused during dismantling, and reinstall the old furniture and/or install new furniture.

In addition, laboratory furniture that has been affixed to the building structure does not facilitate minor rearrangements to accommodate the needs and desires of different individual laboratory workers using the furniture. For example, the height of the bench or the position of storage units under the bench cannot be easily adjusted to accommodate different workers.

U.S. Pat. No. 4,544,214 discloses a laboratory furniture system offering some advantages and improved reconfigurability as compared with conventional permanently affixed laboratory furniture. The furniture system includes individual laboratory stations that are comprised of a combination of adjustable furniture modules. The individual modules may be free standing or connected to one another in an end-to-end relationship, and are adapted to support, at adjustable elevations, standardized shelves, modular drawers, and/or modular cabinetry. However, repositioning of storage units on the modules, and adjustment of the height of the bench mounted on a module require considerable effort, involving manipulation of hardware such as clamps, brackets and the like. Similarly, movement of the modules to another location requires unbolting of the modules from the laboratory floor.

U.S. Pat. No. 6,029,587 discloses a desk supported by a pair of legs having telescoping portions for height adjustability of a horizontal work surface. The support legs include an L-shaped bracket that provides an asymmetric buttress for the work surface with respect to the support legs while maximizing the area under the desk so that a pedestal including a file cabinet can be positioned underneath the desk top.

U.S. Pat. Nos. 3,356,434 and 4,337,988 disclose modular furniture systems in which a furniture module having casters can be rolled adjacent to or underneath a fixture and secured thereto.

U.S. Pat. Nos. 4,050,752; 4,382,642; 4,974,913; and 6,817,684, and European Patent Application No. 0 123 972 A2 disclose fixtures and/or furnishings without casters having suspended storage units.

### SUMMARY OF THE INVENTION

The invention provides a laboratory furniture unit comprising a frame defining horizontally spaced apart rails for supporting at least one storage unit; a laboratory bench supported on the frame; and at least one storage unit slidably supported on the rails below the bench.

The invention also provides a laboratory furniture unit comprising a frame defining horizontally spaced apart rails for supporting at least one storage unit; a laboratory bench supported on the frame; and at least one storage unit releasably supported on the rails below the bench.

These and other features, advantages and objects of the present invention will be further understood and appreciated by those skilled in the art by reference to the following specification, claims and appended drawings.

### BRIEF DESCRIPTION OF THE DRAWINGS

FIG. 1 is a perspective view of a laboratory furniture unit having features in accordance with the invention.

FIG. 2 is a side elevational cross section of the laboratory furniture unit showing the support rails and support brackets for slidably and releasably suspending a storage unit from the frame of the furniture unit.

FIG. 3 is an enlarged perspective view, with the bench removed, showing an adjustable bracket positioned on a front support rail of the furniture unit.

FIG. 4 is an elevational side view showing a powered lift system for adjusting the height of a bench mounted on the furniture unit.

FIG. 5 is a perspective view of the back of a storage unit slidably supportable on rails of the furniture unit, and showing fixed brackets engageable with a rear support rail to support the storage unit on the furniture unit.

### DETAILED DESCRIPTION OF PREFERRED EMBODIMENT

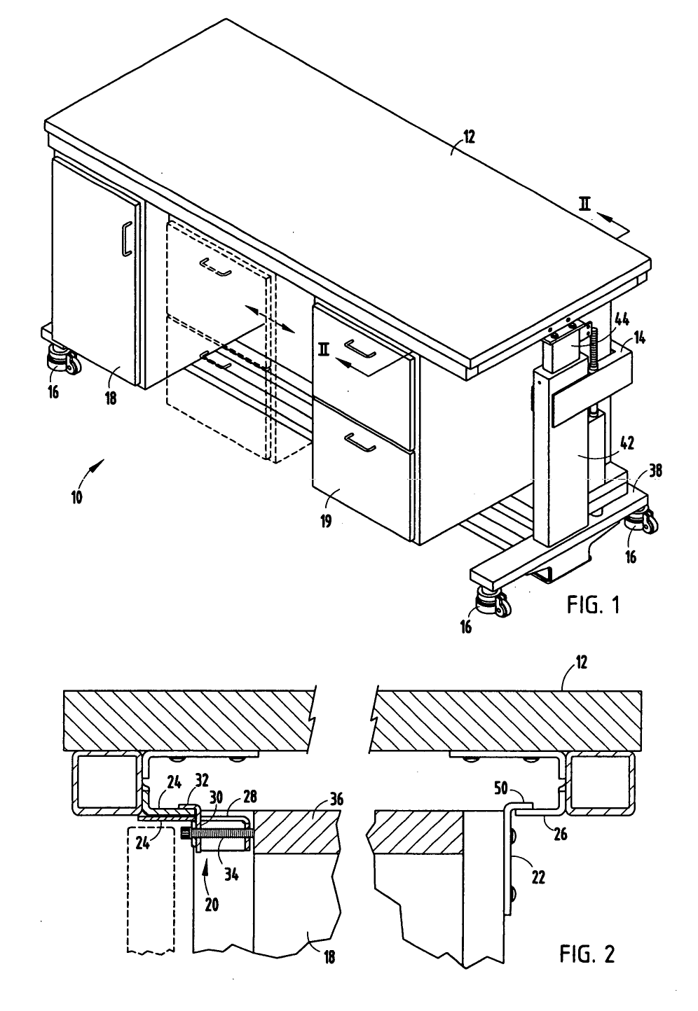

A laboratory furniture unit 10 in accordance with an embodiment of the invention is shown in FIG. 1. Laboratory furniture unit 10 includes a bench 12 defining a horizontal planar laboratory work surface for supporting laboratory equipment and/or other objects. Bench 12 is mounted on an adjustable height frame 14, which allows bench 12 to be raised or lowered as desired. Adjustable height frame 14 is set on a plurality of casters 16, which allow movement of laboratory furniture unit 10 in any desired direction for repositioning of laboratory furniture unit 10. Suspended from adjustable height frame 14 below bench 12 are storage units 18, 19 which may be a cabinet, with or without a closable door, a series of drawers, etc.

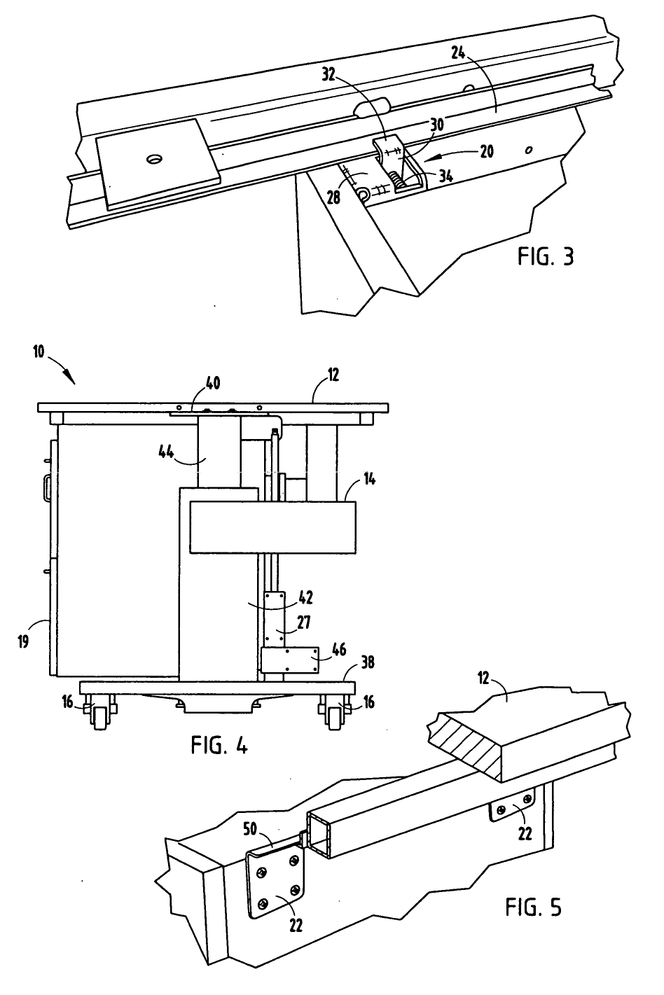

As shown in FIGS. 2 and 3, storage unit 18 includes adjustable brackets 20 attached to a front side of storage unit 18, and fixed brackets 22 attached to a rear side of storage unit 18.

The adjustable brackets 20 are engageable with a front support rail 24, and the fixed brackets 22 are engageable with a rear support rail 26 to facilitate suspension of storage unit 18 from laboratory furniture unit 10 below bench 12. Adjustable bracket 20 includes a stationary member 28 affixed to a top front edge of storage unit 18, and an inverted L-shaped member 30 that is linearly moveable with respect to stationary member 28 between a position in which horizontal extension 32 of inverted L-shaped member 30 rests upon front support rail 24 to suspend storage unit 18 on laboratory furniture unit 10 when fixed brackets 22 are engaged with rear support rail 26, and another position in which horizontal extension 32 is retracted away from front support rail 24 to facilitate removal of storage unit 18 from laboratory furniture unit 10. The relative position of inverted L-shaped member 30 with respect to stationary member 28 of adjustable bracket 20 may be varied by rotation of a screw 34. Installation of a storage unit 18 onto laboratory furniture unit 10 is achieved by retracting L-shaped member 30 toward the front edge of top wall 36 of storage unit 18 to provide sufficient clearance so that fixed brackets 22 can be positioned on rear support rail 26 and storage unit 18 can be rotated into a level position whereby inverted L-shaped member 30 can be moved to an extended position in which horizontal extension 32 is positioned on front support rail 24.

As shown in FIGS. 1 and 4, laboratory furniture unit 10 includes a lower base frame 38, and an upper frame 40 that is moveable or repositionable in an upwardly or downwardly direction with respect to base frame 38. Base frame 38 includes two upright hollow stanchions 42 at opposite ends of base frame 38, and upper frame 40 includes two dependent members 44, each disposed within one of the hollow upright stanchions 42. Upright stanchions 42 define a guide or track for linear vertical movement of dependent members 44 upwardly and downwardly within the hollow space defined in upright stanchions 42.

Upward and downward movement of upper frame 40 with respect to base frame 38 may be achieved by any appropriate means, such as a pneumatic lift system, a hydraulic lift system, or an electronic lift system. Use of an electric motor is preferred because it provides the greatest flexibility, ease of use, and ease of installation. To facilitate smooth travel of upper frame 40 with respect to base frame 38, an electric drive motor 46 is provided for raising each member 44 with respect to stanchions 42. The use of two synchronized motors that operate in tandem to independently raise each of dependent members 44 with respect to each of respective stanchions 42 is desirable to reduce the need for complex mechanical linkages that would ordinarily be employed to raise members 44 simultaneously to prevent binding.

Any of various mechanical devices or linear actuators 27 may be employed for transmitting power from the output shafts of electric motors 46 to dependent members 44 for raising or lowering upper frame 40 with respect to base frame 38. Such mechanical devices include a rack and pinion arrangement, or a screw drive arrangement 27. Suitable tandem twin drive electronic screw lifts capable of lifting a substantial load (at least 2000 pounds) are commercially available and may be advantageously employed in certain embodiments of the invention.

Desirably, the range of height adjustability for bench 12 is selected to accommodate use of laboratory furniture unit 10 by individuals of various heights, as well as seated individuals, including those in wheelchairs. An appropriate height adjustability is from about 30 to about 42 inches for the top surface of bench 12. However, this range of travel may be increased or decreased as desired for particular applications. Preferably, a suitable electronic drive system is selected to provide smooth, uninterrupted travel over the entire range in about 50 seconds or less, by activating an electronic switch located at the front of laboratory furniture unit 10.

Casters 16 typically have a relatively high load bearing capacity (e.g., at least about 500 pounds each), and are desirably provided with drop-down pins that release the weight of the cabinet and any equipment supported in or on the cabinet from the casters to provide a fixed bench position. Generally, each caster 16 comprises a wheel mounted on a swivel. This allows the horizontal wheel axle to be arranged in any direction with respect to laboratory furniture unit 10, thereby facilitating movement of laboratory furniture unit 10 in any direction.

As shown in FIGS. 3 and 5, fixed brackets 22 include horizontal extensions 50 that are positioned on rear support rails 26, and cooperate with horizontal extensions 32 on inverted L-shaped members 30 positioned on front support rail 24 to suspend storage unit 18 on laboratory furniture unit 10 under bench 12. In order to facilitate sliding movement of horizontal extensions 50 and 32 along respective rails 24 and 26, whereby storage unit 18 may be slidably moved along the length of laboratory furniture unit 10 into generally any position between stanchions 42, the underside of horizontal extensions 32 and 50 are provided with an antifriction coating, such as a (UHMW) ultra high molecular weight tape. Alternatively, the under surfaces of horizontal extensions 50 and/or 32 in contact with the upper surfaces of rails 24 and 26, the upper surfaces of support rails 24 and 26 in contact with the under surfaces of horizontal extensions 50 and 32, or both the upward surfaces of support rails 24, 26 and the under surfaces of extensions 50 and 32 may be provided with other antifriction coatings, antifriction surface treatments, and/or antifriction pads or inserts. However, it has been determined that smooth, easy sliding of storage unit 18 can be achieved by providing the upper surfaces of support rails 24 and 26 with smooth surfaces, and by providing the underside of horizontal extensions 32 and 50, which contact upper surfaces of support rails 24 and 26, with a UHMW antifriction tape.

The above description is considered that of the preferred embodiment only. Modifications of the invention will occur to those skilled in the art and to those who make or use the invention. Therefore, it is understood that the embodiments shown in the drawings and described above are merely for illustrative purposes and not intended to limit the scope of the invention, which is defined by the following claims as interpreted according to the principles of patent law, including the doctrine of equivalents.

The invention claimed is:

1. A laboratory furniture unit comprising:
a frame defining horizontally spaced apart rails for supporting at least one storage unit;
a laboratory bench supported on the frame; and
at least one storage unit slidably supported on the rails and below the bench.

2. The laboratory furniture unit of claim 1, further comprising a plurality of casters attached to the frame, whereby the laboratory furniture unit can be rolled along a surface.

3. The laboratory furniture unit of claim 1, wherein the frame comprises a base frame structure and an upper frame structure supported on the base frame structure, the upper frame structure being vertically moveable with respect to the base frame structure.

4. The laboratory furniture unit of claim 3, further comprising a linear actuator for moving the upper frame structure with respect to the base frame structure, and at least one motor mounted on the base frame structure or upper frame structure, the motor having an output shaft linked to the liner actuator to facilitate raising or lowering of the upper frame with respect to the lower frame when the motor is activated.

5. The laboratory furniture unit of claim 4, wherein the motor is an electric motor.

6. The laboratory furniture unit of claim 3, wherein the base structure includes two hollow stanchions, the upper frame structure including two dependent members, each dependent member disposed in one of the stanchions, the stanchions defining a guide for linear vertical movement of the dependent members upwardly and downwardly within the stanchions.

7. The laboratory furniture unit of claim 6, further comprising a linear actuator associated with each of the dependent members, and an electric motor associated with each of the dependent members, the motors being synchronized and operatively connected to the linear actuators to effect raising and lowering of the upper frame structure relative to the lower frame structure.

8. The laboratory furniture unit of claim 1, wherein the storage unit includes at least two fixed brackets engaging one of the spaced apart rails, and at least two adjustable brackets that releasably engage the other rail.

9. The laboratory furniture unit of claim 8, wherein the adjustable bracket includes a stationary member affixed to the storage unit, and an inverted L-shaped member having a horizontal extension engaging one of the support rails, the L-shaped member being repositionable with respect to the stationary member to facilitate releaseability of the storage unit from the laboratory furniture unit.

10. The laboratory furniture unit of claim 8, wherein the surfaces of the brackets contacting the rails are provided with an antifriction coating.

11. The laboratory furniture unit of claim 10, wherein the antifriction coating is a ultra high molecular weight tape.

12. A laboratory furniture unit comprising:
a frame defining horizontally spaced apart rails for supporting at least one storage unit;
a laboratory bench supported on the frame; and
at least one storage unit releasably supported on the rails below the bench.

13. The laboratory furniture unit of claim 12, further comprising a plurality of casters attached to the frame, whereby the laboratory furniture unit can be rolled along a surface.

14. The laboratory furniture unit of claim 12, wherein the frame comprises a base frame structure and an upper frame structure supported on the base frame structure, the upper frame structure being vertically moveable with respect to the base frame structure.

15. The laboratory furniture unit of claim 14, further comprising a linear actuator for moving the upper frame structure with respect to the base frame structure, and at least one motor mounted on the base frame structure or upper frame structure, the motor having an output shaft linked to the liner actuator to facilitate raising or lowering of the upper frame with respect to the lower frame when the motor is activated.

16. The laboratory furniture unit of claim 15, wherein the motor is an electric motor.

17. The laboratory furniture unit of claim 14, wherein the base structure includes two hollow stanchions, the upper frame structure including two dependent members, each dependent member disposed in one of the stanchions, the stanchions defining a guide for linear vertical movement of the dependent members upwardly and downwardly within the stanchions.

18. The laboratory furniture unit of claim 12, wherein the storage unit includes at least two fixed brackets engaging one of the spaced apart rails, and at least two adjustable brackets that releasably engage the other rail.

19. The laboratory furniture unit of claim 18, wherein the adjustable bracket includes a stationary member affixed to the storage unit, and an inverted L-shaped member having a horizontal extension engaging one of the support rails, the L-shaped member being repositionable with respect to the stationary member to facilitate releaseability of the storage unit from the laboratory furniture unit.
